(19) United States
(12) Patent Application Publication
(10) Pub. No.: US 2008/0033173 A1
(43) Pub. Date: Feb. 7, 2008

(54) Method for the Production of Quaternary Ammonia Compounds at Atomospheric Pressure

(75) Inventors: Laszlo Szarvas, Ludwigshafen (DE); Klemens Massonne, Bad Durkheim (DE); Silke Reidl, Hassloch (DE); Walter Saas, Einselthum (DE); Lothar Rub, Speyer (DE); Reiner Kober, Fussgonheim (DE); Berthold Erhart, Bad Durkheim (DE); Steffen Kudis, Mannheim (DE)

Correspondence Address: ROTHWELL, FIGG, ERNST & MANBECK, P.C., 1425 K STREET, N.W., SUITE 800, WASHINGTON, DC, 20005, US

(73) Assignee: BASF AKTIENGESELLSCHAFT, LUDWIGSHAFEN (DE)

(21) Appl. No.: 11/660,087

(22) PCT Filed: Aug. 12, 2005
(86) PCT No.: PCT/EP05/08808

§ 371 Date: Feb. 13, 2007

(30) Foreign Application Priority Data
Aug. 13, 2004 (DE) 10 2004 039 418.0

(51) Int. Cl. C07D 295/02; C07C 209/00

(52) U.S. Cl. 544/229; 564/281

(57) ABSTRACT

The present invention relates to a process for preparing a quaternary ammonium compound, which comprises reacting an amine compound containing at least one sp³-hybridized nitrogen atom with a dialkyl sulfate at ambient pressure with participation of both alkyl groups of the dialkyl sulfate and, if appropriate, subjecting the resulting quaternary ammonium compound containing sulfate anions to an anion exchange.

The present invention relates to a process for preparing a quaternary ammonium compound, which comprises reacting an amine compound which comprises at least one sp³-hybridized nitrogen atom with a dialkyl sulfate at ambient pressure with participation of both alkyl groups of the dialkyl sulfate and, if appropriate, subjecting the resulting quaternary ammonium compound containing sulfate anions to an anion exchange.

Quaternary ammonium compounds are used in large quantities for various applications. Thus, quaternary ammonium compounds having at least one long alkyl chain display surface-active properties and are used as cationic surfactants, e.g. as wetting agents, antistatics, etc. Predominantly short-chain quaternary ammonium compounds have microbicidal properties and are therefore used in fungicidal and bactericidal disinfectants. In organic synthesis, quaternary ammonium compounds are used as phase transfer catalysts. In addition, there are various industrial uses of individual specific quaternary ammonium compounds. Salts made up of quaternary ammonium ions and suitable anions which are liquid at low temperatures (<100° C.) have now become more widely used as ionic liquids.

For possible use in ionic liquids and also for other fields of application, especially in pharmacy and in agriculture, the anion of the quaternary ammonium compound plays a critical role. Thus, the nature of the anion influences important use properties such as the boiling point, the pharmaceutical compatibility, bioavailability, etc. Although there are many known methods of replacing an anion which is not very suitable or unsuitable for a particular application by a more suitable anion, these are frequently complicated and correspondingly expensive. Thus, for example, halide anions have various disadvantages and there is a need for quaternary ammonium compounds which are substantially free of halide anions. Their essentially complete removal to contents which generally do not exceed 1 ppm is difficult because of the corrosive nature of these anions, since many ion-exchange membranes are attacked by these anions. There is therefore a great need for processes which are suitable for the economical preparation of quaternary ammonium compounds which are substantially free of undesirable anions or have an anion which can easily be replaced by another anion.

It is known that amines can be alkylated by means of dialkyl sulfate, but in general only one of the alkyl groups of the dialkyl sulfate is utilized, so that the corresponding monoalkylsulfate salts result. Thus, U.S. Pat. No. 3,366,663 describes a process for preparing tetraalkylammonium alkylsulfates in which a dialkyl sulfate, e.g. dimethyl sulfate, is reacted with a trialkylamine.

EP-A-1 182 196 describes a process for preparing ionic liquids, in which the amines, phosphines, imidazoles, pyridines, triazoles or pyrazoles on which the cation is based are alkylated by means of a dialkyl sulfate to give salts of the corresponding monoalkylsulfate anions and these are subsequently subjected to an anion exchange with metal salts.

WO 02/12179 describes a process for the sulfation of compounds having hydroxyl groups. Here, an ammonium monoorganylsulfate formed from a tertiary amine and a diorganyl sulfate is used as sulfating agent.

In J. Chem. Soc., Chem. Commun., 1992, pp. 965-967, J. S. Wilkes and M. J. Zaworotko describe ionic liquids based on a 1-ethyl-3-methylimidazolium cation. Starting from the iodide compound, further anions, e.g. the sulfate in the form of its monohydrate, can be prepared by anion exchange with the corresponding silver salts.

WO 03/074494 describes halogen-free ionic liquids based on anions of the formula [R′—O—SO₃]⁻ or [R′—SO₃]⁻, where R′ is a group of the general formula R⁵—[X(—CH₂—)ₙ]ₘ in which n is from 1 to 12, m is from 1 to 400, X is oxygen, sulfur or a group of the general formula —O—Si(CH₃)₂—O—, —O—Si(CH₂CH₃)₂—O—, —O—Si(OCH₃)₂—O— or —O—Si(O—CH₂CH₃)₂—O— and R⁵ is an optionally functionalized alkyl group. They are prepared from pyridine-SO₃ complexes and ethers of the formula R′—OH.

The Belgian patent BE 750 372 describes a process for preparing uncharged quaternary ammonium salts of polybasic acids, in which a quaternary ammonium salt of an acid ester of a polybasic acid, e.g. a tetraalkylammonium alkylsulfate, is hydrolyzed and subsequently treated with an alkali metal hydroxide.

JP-A-57 126465 describes a process for preparing tetraalkylammonium salts, in which a tetraalkylammonium alkylsulfate, e.g. tetraethylammonium ethylsulfate, is treated with an anion exchanger containing OH⁻ anions and the resulting tetraalkylammonium hydroxide is neutralized with an acid.

DE-A-15 43 747 (U.S. Pat. No. 3,371,117) describes a process for the direct preparation of a bisquaternary ammonium salt from a dialkyl sulfate ester and a trialkylamine by reaction at a temperature in the range from 0 to 400° C. and a pressure which is sufficient to prevent vaporization of the amine. Since hydrolysis of the sulfate ester has to take place at elevated temperatures, this document teaches carrying out the reaction in two stages, with one alkyl group of the sulfate ester initially participating in the alkylation at a low temperature in the range from about 0 to 50° C. and the second alkyl group then participating in a second step at elevated temperature in the range from about 50 to 400° C.

WO 99/09832 describes plant growth regulators comprising a quaternary N-methyl-piperidinium salt (Mepiquat salt) and a water-soluble boron salt.

WO 99/52368 describes a Mepiquat plant growth regulator composition having a boron-containing anion. To prepare it, a Mepiquat chloride can first be converted electrochemically into Mepiquat hydroxide and subsequently be reacted with boric acid. Furthermore, it can be prepared by reacting Mepiquat hydroxide, hydrogencarbonate or carbonate with boric acid or appropriate salts of boric acid. The carbonates or hydrogencarbonates used as starting material can be obtained by quaternization of N-methylpiperidine with dimethyl carbonate. A disadvantage of this reaction is that it is generally carried out at elevated temperatures and under superatmospheric pressure.

The unpublished German patent application 10 2004 010 662.2 describes a process for preparing ionic compounds comprising cations having quaternary sp²-hybridized nitrogen atoms, in which compounds comprising a double-bonded nitrogen atom are reacted with a dialkyl sulfate at elevated temperature with participation of both alkyl groups of the dialkyl sulfate and, if appropriate, the resulting ionic compound containing sulfate anions is subjected to an anion exchange.

The unpublished German patent application 10 2004 026 153.9 describes a process for preparing a quaternary ammonium compound, in which an amine compound which comprises at least one sp³-hybridized nitrogen atom is reacted with a dialkyl sulfate or trialkyl phosphate to give a quaternary ammonium compound which has at least some polyvalent anions and this is subsequently subjected to an anion exchange.

None of the abovementioned processes describes a quaternization of amines by means of dialkyl sulfates in which both alkyl groups of the dialkyl sulfate are utilized and the reaction is carried out at ambient pressure.

It is an object of the present invention to provide a simple and thus economical process for preparing quaternary ammonium compounds. In particular, the process should be suitable for the preparation of quaternary ammonium compounds which are substantially free of undesirable anions, especially halides, or have anions which can be replaced in a simple manner.

We have accordingly found a process for preparing a quaternary ammonium compound, which comprises
a) reacting an amine compound which comprises at least one sp³-hybridized nitrogen atom and has a boiling point under normal conditions of at least 80° C. with a dialkyl sulfate at ambient pressure with participation of both alkyl groups of the dialkyl sulfate to give a quaternary ammonium compound containing sulfate anions, and
b) if appropriate, subjecting the quaternary ammonium compound obtained in step a) to an anion exchange.

It has surprisingly been found that nonvolatile or only slightly volatile amine compounds (boiling point≧80° C. at 101325 Pa) which have at least one sp³-hybridized nitrogen atom can be quaternized by means of dialkyl sulfates with participation of both alkyl groups even at ambient pressure. Quaternary ammonium compounds having doubly negatively charged sulfate anions as anion component are advantageously obtained in this way. Firstly, the alkyl group equivalents of the dialkyl sulfate can be utilized effectively, and, secondly, the sulfate compounds obtained are potentially interesting active compounds, e.g. for use in crop protection and as growth regulators for plants, and are also good intermediates for subsequent anion exchange. The process of the invention is also particularly useful for preparing halide-free quaternary ammonium compounds. Despite the reaction at ambient pressure, the hydrolysis described in the prior art as a disadvantage in the double alkylation by means of dialkyl sulfate is advantageously not observed.

In a specific embodiment of the process of the invention, the quaternary ammonium compound obtained in step a) is additionally subjected to at least one work-up step to separate off unreacted amine compounds. Unreacted amine compounds are present in detectable amounts in the reaction product obtained in step a) especially when, as described below, a molar excess of amine equivalents over sulfate equivalents (i.e. a molar ratio of amine compound to dialkyl sulfate of >2:1) is used. The amine compound used in step a) is then preferably an amine compound which forms a low-boiling azeotrope with water, and the reaction in step a) is carried out in water or an aqueous medium. Unreacted amine compound can then be separated off in a simple manner by azeotropic distillation. Amine compounds which form a low-boiling azeotrope with water can also be separated off from nonaqueous solvents by steam distillation, i.e. by addition of heated water or passage of hot steam through the reaction mixture during the distillation.

For the purposes of the present invention, “ambient pressure” is the pressure established in the reaction vessel when it is not closed off from the environment in a pressure-tight manner. The ambient pressure varies with the prevailing atmospheric pressure and the room temperature. It is generally in the region of atmospheric pressure of 101 325 Pa, i.e., for example, in a range from 95 000 to 110 000 Pa. The expression “ambient pressure” also refers to the pressure in the reaction vessel which is established when a reactant gas or inert gas is introduced into the reaction vessel without a significant overpressure being able to occur (e.g. by simple passage through the apparatus which is not closed in a gas tight manner). The ambient pressure thus does not correspond to the autogenous pressure which is established when working in pressure-tight apparatuses, e.g. in autoclaves. For the purpose of explaining the present invention, the expression “alkyl” encompasses straight-chain and branched alkyl groups. It preferably refers to straight-chain or branched C₁-C₂₀-alkyl, preferably C₁-C₁₀-alkyl groups, particularly preferably C₁-C₈-alkyl groups and very particularly preferably C₁-C₄-alkyl groups. Examples of alkyl groups are, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression “alkyl” also encompasses substituted alkyl groups which generally have 1, 2, 3, 4 or 5 substituents, preferably 1, 2 or 3 substituents and particularly preferably 1 substituent. These are, for example, selected from among cycloalkyl, aryl, hetaryl, halogen, amino, alkoxycarbonyl, acyl, nitro, aminocarbonyl, alkylaminocarbonyl, dialkylaminocarbonyl, alkylcarbonylamino, carboxylate and sulfonate.

The expression “alkylene” as used for the purposes of the present invention refers to straight-chain or branched alkanediyl groups which preferably have from 1 to 5 carbon atoms.

The expression “cycloalkyl” as used for the purposes of the present invention encompasses both unsubstituted and substituted cycloalkyl groups, preferably C₅-C₈-cycloalkyl groups, e.g. cyclopentyl, cyclohexyl or cycloheptyl. If they are substituted, these can generally bear 1, 2, 3, 4 or 5 substituents, preferably 1, 2 or 3 substituents. These substituents are, for example, selected from among alkyl and the substituents mentioned above for substituted alkyl groups.

The expression “heterocycloalkyl” as used for the purposes of the present invention encompasses saturated, cycloaliphatic groups which generally have from 4 to 7, preferably 5 or 6 ring atoms and in which 1, 2, 3 or 4 of the ring carbons are replaced by heteroatoms selected from among the elements oxygen, nitrogen and sulfur and which may optionally be substituted. If they are substituted, these heterocycloaliphatic groups can bear, for example, 1, 2 or 3 substituents. These substituents are, for example, selected from among alkyl and the substituents mentioned above for substituted alkyl groups. Examples of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

The expression “aryl” as used for the purposes of the present invention encompasses both unsubstituted and substituted aryl groups, and preferably refers to phenyl, tolyl, xylyl, mesityl, naphthyl, fluorenyl, anthracenyl, phenanthrenyl or naphthacenyl, particularly preferably phenyl or naphthyl. If they are substituted, these aryl groups can generally bear 1, 2, 3, 4 or 5 substituents, preferably 1, 2 or 3 substituents. These substituents are, for example, selected from among alkyl and the substituents mentioned above for substituted alkyl groups.

The expression “hetaryl” as used for the purposes of the present invention encompasses unsubstituted or substituted, heterocycloaromatic groups, preferably the groups pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl. If they are substituted, these heterocycloaromatic groups can generally have 1, 2 or 3 substituents. These substituents are, for example, selected from among alkyl and the substituents mentioned above for substituted alkyl groups.

For the purposes of the present invention, carboxylate and sulfonate are preferably derivatives of a carboxylic acid function or a sulfonic acid function, in particular a metal carboxylate or sulfonate, a carboxylic ester or sulfonic ester function or a carboxamide or sulfonamide function. They include, for example, esters with C₁-C₄-alkanols such as methanol, ethanol, n-propanol, isopropanol, n-butanol, sec-butanol and tert-butanol.

The above explanations of the expressions “alkyl”, “cycloalkyl”, “aryl”, “heterocycloalkyl” and “hetaryl” apply analogously to the expressions “alkoxy”, “cycloalkoxy”, “aryloxy”, “heterocycloalkoxy” and “hetaryloxy”.

The expression “acyl” as used for the purposes of the present invention refers to alkanoyl or aroyl groups which generally have from 2 to 11, preferably from 2 to 8, carbon atoms, for example the acetyl, propanoyl, butanoyl, pentanoyl, hexanoyl, heptanoyl, 2-ethylhexanoyl, 2-propylheptanoyl, benzoyl or naphthoyl group.

The groups NE¹E² are preferably N,N-dimethylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Halogen is fluorine, chlorine, bromine and iodine, preferably fluorine, chlorine and bromine.

M⁺ is a cation equivalent, i.e. a monovalent cation or the fraction of a polyvalent cation corresponding to a single positive charge. The cation M⁺ serves merely as counterion to balance the charge of negatively charged substituent groups such as COO⁻ or the sulfonate group and can in principle be chosen at will. Preference is therefore given to using alkali metal ions, in particular Na⁺, K⁺, Li⁺ ions, or onium ions such as ammonium, monoalkylammonium, dialkylammonium, trialkylammonium, tetraalkylammonium, phosphonium, tetraalkylphosphonium or tetraarylphosphonium ions.

An analogous situation applies to the anion equivalent A⁻ which serves merely as counterion for positively charged substituent groups such as the ammonium groups and can be chosen at will among monovalent anions and the fractions of a polyvalent anion corresponding to a single negative charge, with preference generally being given to anions other than halide ions.

For the purposes of the present invention, the term polycyclic compounds encompasses in the widest sense compounds which comprise at least two rings, regardless of whether these rings are linked. The rings can be carbocyclic and/or heterocyclic. The rings can be linked via single or double bonds (“multinuclear compounds”), joined by fusion (“fused ring systems”) or bridged (“bridged ring systems”, “cage compounds”). Fused ring systems can be (fused-on) aromatic, hydroaromatic and cyclic compounds linked by fusion. Fused ring systems have two, three or more than three rings. Depending on the way in which the rings are linked, a distinction is made in the case of fused ring systems between ortho-fusion, i.e. each ring shares an edge or two atoms with each adjacent ring, and peri-fusion in which a carbon atom belongs to more than two rings. The bridged ring systems include, for the purposes of the present invention, those which do not belong to the multinuclear ring systems and fused ring systems and in which at least two ring atoms belong to at least two different rings. In the case of the bridged ring systems, a distinction is made, depending on the number of ring opening reactions formally required to obtain an open-chain compound, between bicyclo, tricyclo, tetracyclo compounds, etc., which comprise two, three, four, etc. rings. The bridged ring systems can, if desired, additionally have, depending on size, one, two, three or more than three fused-on rings.

Advantageously, the quaternary ammonium compounds containing sulfate anions which are obtained in step a) of the process of the invention are suitable for a subsequent single-stage or multistage anion exchange.

The process of the invention is quite generally suitable for the preparation of ionic compounds of the formula I
bCat^{m+}×Xⁿ⁻ (I)
where
Cat^{m+} is an m-valent cation containing at least one quaternary sp³-hybridized nitrogen atom,
Xⁿ⁻ is an n-valent anion,
b and x are integers≧1, with the proviso that (b times m)=(x times n).

Compounds of this type include compounds of the formulae Cat⁺X⁻, Cat^{m+}X^{m-}, n Cat⁺ Xⁿ⁻ and Cat^{m+}mX⁻, where m and n are integers>1.

The anion component Xⁿ⁻ is preferably an anion other than Cl⁻, Br⁻, I⁻ and monoalkylsulfates and monoalkylphosphates. The anions Xⁿ⁻ are preferably selected from among hydroxide (OH⁻), sulfate (SO₄²⁻), hydrogensulfate (HSO₄⁻), nitrite (NO₂⁻), nitrate (NO₃⁻), cyanide (CN⁻), cyanate (OCN⁻), isocyanate (NCO⁻), thiocyanate (SCN⁻), isothiocyanate (NCS⁻), phosphate (PO₄³⁻), hydrogenphosphate (HPO₄²⁻), dihydrogenphosphate (H₂PO₄⁻), primary phosphite (H₂PO₃⁻), secondary phosphite (HPO₃²⁻), hexafluorophosphate ([PF₆]⁻), hexafluoroantimonate ([SbF₆]⁻), hexafluoroarsenate ([AsF₆]⁻), tetrachloroaluminate ([AlCl₄]⁻), tetrabromoaluminate ([AlBr₄]⁻), trichlorozincate ([ZnCl₃]⁻), dichlorocuprates (I) and (II), carbonate (CO₃²⁻), hydrogencarbonate (HCO₃⁻), fluoride (F⁻), triorganylsilanolate R′₃SiO⁻, fluorosulfonate (R′—COO)⁻, sulfonate (R′—SO₃) and [(R′—SO₂)₂N]⁻, where R′ is alkyl, cycloalkyl or aryl. R is preferably a linear or branched aliphatic or alicyclic alkyl radical comprising from 1 to 12 carbon atoms or a C₅-C₁₈-aryl, C₅-C₁₈-aryl-C₁-C₆-alkyl or C₁-C₆-alkyl-C₅-C₁₈-aryl radical which may be substituted by halogen atoms.

For use in the agriculture sector, e.g. as or in crop protection agent(s), plant growth regulator(s), etc., preference is given to quaternary ammonium compounds which are substantially free of anions which are of no use or little use to plants, in particular halides. In this sector, preference is given to using anions Xⁿ⁻ selected from among sulfate, hydrogensulfate, nitrate, phosphate and boron-containing anions.

In a particularly preferred embodiment, the ionic compound obtained by the process of the invention is a compound based on a boron-containing anion. The term “quaternary ammonium compound” as is used for the purposes of the present invention encompasses both “salts” and “coordination compounds” or “complexes”. The term “ions” also encompasses “complex ions”. The distinctions made in this respect in the case of boron compounds in some other documents do not apply for the purposes of the present invention.

For the purposes of the present invention, the term “borate salt” encompasses salts, coordination compounds and complexes comprising borate anions. The term also encompasses mixed anion species which comprise at least one borate anion and at least one different anion. The term “borate” encompasses both hydrated and anhydrous anion species based on boron-oxygen compounds including chain and ring structures, oligomorphic and polymorphic forms, etc. A person skilled in the art will know that the structure of borate anions or polyanions varies as a function of the chemical environment, e.g. depending on whether the compound is present as a solid or in solution, and also, for example, as a function of the pH of the solvent. In the following, B represents boron and O represents oxygen, in accordance with customary nomenclature.

The anion component Xⁿ⁻ is preferably a boron-containing anion which is selected from among anions of the general formula II
[MₓB_{y}O_{z}(A)ᵥ]ⁿ⁻.wH₂O (II)
where

M is hydrogen, NH₄ or a different agriculturally acceptable cation,

A is a ligand,

n is an integer in the range from 1 to 6,

x is an integer or fraction in the range from 0 to 10,

y is an integer or fraction in the range from 1 to 48,

z is an integer or fraction in the range from 0 to 48,

v is an integer or fraction in the range from 0 to 24, and

w is an integer or fraction in the range from 0 to 24.

Suitable agriculturally acceptable cations are, for example, Na, K, Mg, Ca, Zn, Mn, Cu and combinations thereof.

The water molecules in the formula II can be free or coordinated water of crystallization or be water which has condensed onto the borate anion and is bound, for example, in the form of hydroxy groups. Suitable values of w are, for example, 0.5; 1; 1.5; 2, 2.5; 3; 4; 5; 6; 7; 8; 10; 12; 20. A preferred value of w is 0.5.

Suitable ligands A) have one or more groups which are capable of association with at least one boron atom and/or an agriculturally acceptable cation. The ligands A) are preferably electron donors. Depending on the type and number of the groups capable of association, simple complexes or chelates result. Any agriculturally acceptable metals present can additionally participate in the formation of the coordinate bond, e.g. via a donor-acceptor interaction. The component A) is preferably selected from among or derived from 1-hydroxycarboxylic acids such as lactic acid, mandelic acid or malic acid; monohydroxy- or oligohydroxy-monocarboxylic, -dicarboxylic or -tricarboxylic acids, e.g. tartaric acid or citric acid; glycols, preferably vicinal glycols such as 1,2-propylene glycol, 2,3-butylene glycol; alcohols such as methanol, ethanol, n-propanol, isopropanol, n-butanol, tert-butanol, pentanol or benzyl alcohol; monocarboxylic, dicarboxylic or tricarboxylic acids such as acetic acid, oxalic acid or benzoic acid; amino alcohols such as ethanolamine or diethanolamine; polyols, sugars and their derivatives, e.g. sugar alcohols, polyhydroxycarboxylic acids, e.g. glycerol, sorbitol, mannitol, glucose, fructose, glucoronic acid; derivatives of the abovementioned compounds, e.g. ethers or esters, which are capable of coordinating to a boron atom, e.g. ethers and esters which have at least one additional group which is capable of coordination and is, for example, selected from among amino, hydroxy and carboxyl groups.

The anions Xⁿ⁻ are preferably selected from among boron-containing anions of the general formula III
[B_{y}O_{z}(A)ᵥ]ⁿ⁻.w(H₂O) (III)
where

A is as defined above,

n is an integer in the range from 1 to 6,

y is an integer or fraction in the range from 1 to 48,

z is an integer or fraction in the range from 0 to 48,

v is an integer or fraction in the range from 0 to 24, and

w is an integer or fraction in the range from 0 to 24.

Preference is given to compounds of the formula III in which y is an integer or fraction in the range from 2 to 20, particularly preferably in the range from 2 to 10, in particular in the range from 3 to 10.

In a further preferred embodiment, the anion Xⁿ⁻ is selected from among compounds of the general formula IV
[MₓB_{y}O_{z}(A)ᵥ]ⁿ⁻.w(H₂O) (IV)
where

M is as defined above,

A is as defined above,

n is an integer in the range from 1 to 6,

x is an integer or fraction in the range from 0 to 10,

y is an integer or fraction in the range from 1 to 48,

z is an integer or fraction in the range from 0 to 48,

v is an integer or fraction in the range from 0 to 24, and

w is an integer or fraction in the range from 0 to 24.

Preference is given to compounds of the formula IV in which y is an integer or fraction in the range from 2 to 20, particularly preferably in the range from 2 to 10, in particular in the range from 3 to 10.

Preference is also given to the anion Xⁿ⁻ being selected from among anions of the formulae II and III in which

y is an integer or fraction in the range from 3 to 7,

z is an integer or fraction in the range from 6 to 10,

v is 0, and

w is an integer or fraction in the range from 0.5 to 10.

The anion Xⁿ⁻ is particularly preferably an anion of the formula III in which

y is an integer or fraction in the range from 3 to 5,

z is an integer or fraction in the range from 3 to 6,

v is 0, and

w is an integer or fraction in the range from 0.5 to 8.

Preference is also given to anions Xⁿ⁻ of the general formula III in which

y is 5,

z is 8,

v is 0, and

w is an integer or fraction in the range from 0.5 to 3.

Quaternary ammonium compounds which are obtainable by the process of the invention and comprise at least one N,N-dimethylpiperidinium cation and at least one of the above-described boron-containing anions are advantageous for use in compositions for regulating plant growth. Such formulations and processes of preparing them are described in WO 99/09832 and WO 99/52368, which are hereby fully incorporated by reference.

Boron-containing anions suitable for the abovementioned use and for further uses are the following: orthoborate (BO₃³⁻), metaborate ((BO₂)₃³⁻), pentaborate B₅O₈⁻, pentaborate hydrate (B₅H₄O₁₀⁻), [B₅O₆(OH)₄]⁻, tetrafluoroborate ([BF₄]⁻), tetrachloroborate ([BCl₄]⁻), tetraphenylborate ([B(C₆H₅)₄]⁻) and hydrates and mixtures thereof.

The amine compound used in step a), which comprises at least one sp³-hybridized nitrogen atom, can be an acyclic or cyclic compound. The cation component Cat^{m+} is derived from these amines by quaternization.

Suitable amine compounds have at least one primary, secondary or tertiary amino function. They are preferably selected from among compounds of the general formula NR¹R²R³, where R¹ R² and R³ are selected independently of one another from among hydrogen, alkyl, cycloalkyl, heterocycloalkyl, aryl and hetaryl, with at least two of the radicals R¹ R² and R³ together with the N atom to which they are bound also being able to be part of a polycyclic compound. Particular preference is given to tertiary amines.

The radicals R¹ R² and R³ are preferably selected independently from among hydrogen, C₁-C₃₀-alkyl, C₃-C₈-cycloalkyl, C₃-C₈-heterocycloalkyl, C₁-C₁₄-aryl and C₁-C₁₄-heteroaryl.

When at least one of the radicals R¹ to R³ is alkyl, it is preferably a C₁-C₂₀-alkyl radical which, as defined above, may be substituted and/or interrupted by 1, 2, 3 or more than 3 nonadjacent heteroatoms or heteroatom-containing groups. The heteroatoms and heteroatom-containing groups are preferably selected from among O, S, NR⁴ and PR⁵, where R⁴ and R⁵ are each, independently of one another, hydrogen, alkyl, cycloalkyl, heterocycloalkyl, aryl, hetaryl, COOR^{a}, COO⁻M⁺, SO₃R^{a}, SO₃⁻M⁺, sulfonamide, NE¹E², (NE¹E²E³)⁺A⁻, OR^{a}, SR^{a}, (CHR^{b}CH₂O)_{y}R^{a}, (CH₂O)_{y}R^{a}, (CH₂CH₂NE¹)_{y}R^{a}, alkylaminocarbonyl, dialkylaminocarbonyl, alkylcarbonylamino, halogen, nitro, acyl or cyano, where
the radicals R^{a} are identical or different and are selected from among hydrogen, alkyl, cycloalkyl, aryl, heterocycloalkyl and hetaryl,
E¹, E², E³ are identical or different radicals selected from among hydrogen, alkyl, cycloalkyl, aryl and hetaryl,
R^{b} is hydrogen, methyl or ethyl,
M⁺ is a cation equivalent,
A⁻ is an anion equivalent and
y is an integer from 1 to 250.

Suitable radicals R¹ to R³ are, for example, hydrogen, methyl, ethyl, n-propyl, sec-propyl, isopropyl, n-butyl, isobutyl, sec-butyl, tert-butyl, n-pentyl and n-hexyl, lauryl, tridecyl, myristyl, palmityl and stearyl. Further suitable radicals R¹ to R³ are 5-, 6- and 7-membered saturated, unsaturated or aromatic carbocycles and heterocycles, e.g. cyclopentyl, cyclohexyl, phenyl, tolyl, xylyl, cycloheptanyl, naphthyl, tetrahydrofuranyl, tetrahydropyranyl, dioxanyl, pyrrolidyl, piperidyl, pyridyl and pyrimidyl.

Suitable amine compounds having a primary amino function are, for example, methylamine, ethylamine, n-propylamine, isopropylamine, n-butylamine, isobutylamine, sec-butylamine, tert-butylamine, pentylamine, hexylamine, cyclopentylamine, cyclohexylamine, aniline and benzylamine.

Suitable amine compounds which have a primary amino function and in which one of the radicals R¹ to R³ is an alkyl radical interrupted by 0 are, for example, CH₃—O—C₂H₄—NH₂, C₂H₅—O—C₂H₄—NH₂, CH₃—O—C₃H₆—NH₂, C₂H₅—O—C₃H₆—NH₂, n-C₄H₉—O—C₄H₈—NH₂, HO—C₂H₄—NH₂, HO—C₃H₇—NH₂ and HO—C₄H₈—NH₂.

Suitable amine compounds having a secondary amino function are, for example, dimethylamine, diethylamine, methylethylamine, di-n-propylamine, diisopropylamine, diisobutylamine, di-sec-butylamine, di-tert-butylamine, dipentylamine, dihexylamine, dicyclopentylamine, dicyclohexylamine and diphenylamine.

Suitable amine compounds which have a suitable amino function and in which one or two of the radicals R¹ to R³ is/are an alkyl radical interrupted by 0 are, for example, (CH₃—O—C₂H₄)₂NH, (C₂H₅—O—C₂H₄)₂NH, (CH₃—O—C₃H₆)₂NH, (C₂H₅—O—C₃H₆)₂NH, (n-C₄H₉—O—C₄H₈)₂NH, (HO—C₂H₄)₂NH, (HO—C₃H₆)₂NH and (HO—C₄H₈)₂NH.

Suitable amine compounds having a tertiary amino function are, for example, trimethylamine, triethylamine, tri(n-propyl)amine, tri(isopropyl)amine, tri(n-butyl)amine, tri(isobutyl)amine, tri(tert-butyl)amine, etc.

Further suitable amine compounds having a tertiary amino function are dialkylarylamines, preferably di(C₁-C₄-)alkylarylamines, in which the alkyl groups and/or the aryl group may be additionally substituted. The aryl group is preferably phenyl. Such amine compounds include, for example, N,N-dimethylaniline, N,N-diethylaniline, N,N,2,4,6-pentamethylaniline, bis(4-(N,N-dimethylamino)phenyl)methylene, 4,4′-bis(N,N-dimethylamino)benzophenone, etc.

Further suitable amine compounds having a tertiary amino function are alkyldiarylamines, preferably (C₁-C₄-)alkyldiarylamines, in which the alkyl group and/or the aryl groups may be substituted. Such amine compounds include, for example, diphenylmethylamine and diphenylethylamine.

Further suitable amine compounds having a tertiary amino function are triarylamines, in which the aryl groups may be substituted, e.g. triphenylamine, etc. Other preferred amines are tricycloalkylamines such as tricyclohexylamine.

When at least two of the radicals R¹ R² and R³ together with the N atom to which they are bound are part of a polycyclic compound, preference is given to two of the radicals R¹ R² and R³ together with the N atom to which they are bound forming an optionally substituted 5- to 7-membered heterocycle which can contain one, two or three further heteroatoms or heteroatom-containing groups selected from among O, S, NR⁴ and PR⁵, where R⁴ and R⁵ are as defined above. Suitable cyclic amine compounds are, for example, pyrrolidine, piperidine, morpholine and piperazine and also their substituted derivatives. Suitable derivatives of the abovementioned nitrogen-containing heterocycles can, for example, have one or more C₁-C₆-alkyl substituents such as methyl, ethyl, n-propyl, isopropyl, n-butyl, sec-butyl, tert-butyl, etc. They include, for example, the N—C₁-C₆-alkyl derivatives. A particularly preferred cyclic tertiary amine is N-methylpiperidine.

Preference is also given to the radicals R¹ R² and R³ together with the N atom to which they are bound forming a bicyclic trialkylenamine or trialkylenediamine, e.g. 1-azabicyclo[2.2.2]octane or 1,4-diazabicyclo[2.2.2]octane.

Further suitable amine compounds are alkylenediamines, dialkylenetriamines, trialkylenetetramines and polyalkylenepolyamines such as oligoalkylenimines or polyalkylenimines, in particular oligoethylenimines or polyethylenimines, preferably oligoethylenimines having from 2 to 20, preferably from 2 to 10 and particularly preferably from 2 to 6, ethylenimine units. Suitable compounds of this type are, in particular, N-propylenediamine, 1,4-butanediamine, 1,6-hexanediamine, diethylenetriamine, triethylenetetramine and polyethyleneimines, and also their alkylation products which have at least one primary or secondary amino function, e.g. 3-(dimethylamino)-n-propylamine, N,N-dimethylethylenediamine, N,N-diethylethylenediamine and N,N,N′,N′-tetramethyldiethylenetriamine. Ethylenediamine is likewise suitable.

Further suitable amine compounds are the reaction products of alkylene oxides, in particular ethylene oxide and/or propylene oxide, with primary and secondary amines.

The abovementioned amine compounds are preferably used individually. However, they can also be used in the form of any mixtures.

To prepare ionic compounds comprising at least one cation containing a quaternary sp³-hybridized nitrogen atom according to the invention, a compound which comprises an sp³-hybridized nitrogen atom is reacted with a dialkyl sulfate in a first reaction step a) to give a quaternary ammonium compound having essentially sulfate anions and, if appropriate, the ionic compound obtained in step a) is subsequently subjected to an anion exchange in a step b).

The reaction in step a) is preferably carried out at an elevated temperature, i.e. at a temperature above ambient temperature. The temperature in step a) is preferably at least 40° C., particularly preferably at least 60° C. The reaction in step a) is preferably carried out at a temperature in the range from 40 to 120° C., particularly preferably from 60 to 100° C.

In a preferred embodiment, step a) is carried out by initially bringing the amine compound into contact with the dialkyl sulfate at a temperature of not more than 35° C. and subsequently heating the resulting mixture to a temperature of at least 40° C. to bring about the further reaction, as described above. If desired, the amine compound can also be brought into contact with the dialkyl sulfate at lower temperatures, e.g. at a temperature of not more than 20° C., especially at a temperature of not more than 10° C. The amine compound is preferably brought into contact with the dialkyl sulfate a little at a time. For this purpose, the amine or the dialkyl sulfate can be placed in a reaction vessel and the respective other component can be added a little at a time. Preference is given to using both components in liquid form, e.g. in the form of an aqueous solution. For the purposes of the present invention, aqueous solutions include water and mixtures of water with water-miscible solvents.

According to the invention, the reaction in step a) is carried out at ambient pressure. The costly use of pressure-rated reactors such as autoclaves can thus advantageously be dispensed with. Suitable reactors for reactions under ambient pressure are known to those skilled in the art and include, for example, stirred reactors which may, if desired, be provided with an internal lining. Furthermore, the use of a condenser can be advantageous, in particular when temperature ranges which are in the region of the boiling point or above the boiling point of the amine compound used are employed.

The molar ratio of the amine compound which is to be alkylated to the dialkyl sulfate is preferably at least 2:1. The molar ratio of the amine compound to be alkylated to the dialkyl sulfate is particularly preferably in a range from 1.8:1 to 10:1, in particular from 2.05:1 to 5:1, especially from 2.1:1 to 3:1.

The reaction of the amine compound with the dialkyl sulfate can be carried out in the absence or preferably in the presence of a solvent which is inert under the reaction conditions. Suitable solvents are, for example, water, water-miscible solvents, for example alcohols such as methanol and ethanol, and mixtures thereof. Preference is given to using water or a solvent mixture comprising at least 30% by volume, preferably at least 50% by volume, in particular at least 80% by volume, of water as solvent.

The dialkyl sulfates used in step a) are preferably di-C₁-C₁₀-alkyl sulfates and, in particular, di-C₁-C₆-alkyl sulfates such as dimethyl sulfate, diethyl sulfate, di-n-propyl sulfate, diisopropyl sulfate, di-n-butyl sulfate, diisobutyl sulfate, di-tert-butyl sulfate, di-n-pentyl sulfate, diisopentyl sulfate, dineopentyl sulfate and di-n-hexyl sulfate. Particular preference is given to using dimethyl sulfate and diethyl sulfate.

If desired, the reaction in step a) can be carried out in the presence of at least one inert gas. Suitable inert gases are, for example, nitrogen, helium and argon. The inert gases are preferably introduced by passing them into the liquid reaction mixture, with the proviso that a reaction pressure higher than ambient pressure does not result.

The quaternary ammonium compounds obtained in step a) can, as described above, be subjected to a work-up step to separate off unreacted amine compound. This is preferably an azeotropic distillation or a steam distillation, depending on whether an aqueous solvent or a nonaqueous solvent is used for the reaction in step a).

The reaction in step a) can be carried out continuously or batchwise.

The quaternary ammonium salts can be isolated from the reaction mixture obtained in step a) by customary methods known to those skilled in the art. This is done especially when the reaction in step b) is to be carried out in a different solvent than is the alkylation in step a). If a solvent has been used for the reaction in step a), this can be removed by evaporation, preferably under reduced pressure. Since the ionic compounds obtained are nonvolatile, the pressure range employed is generally not critical. If a virtually complete removal of the solvent is desired, it is possible to employ, for example, a fine vacuum of from 10¹ to 10⁻¹ Pa or a high vacuum of from 10⁻¹ to 10⁻⁵ Pa. To generate the pressure, it is possible to use customary vacuum pumps such as liquid jet vacuum pumps, rotary vane and rotary piston vacuum pumps, diaphragm vacuum pumps, diffusion pumps, etc. The removal of the solvent can also be carried out at an elevated temperature of up to 150° C., preferably up to 100° C.

The reaction mixture obtained in step a) is preferably used for the reaction in step b) without prior isolation.

The anion exchange in step b) can be effected by transprotonation, reaction with a metal salt, ion exchange chromatography, electrolytically or a combination of these measures.

In a first embodiment, the quaternary ammonium compound obtained in step a) of the process of the invention, which has at least some polyvalent anions, is reacted with an acid, preferably sulfuric acid or phosphoric acid, with proton transfer.

To carry out the transprotonation, a quaternary ammonium compound having sulfate anions is preferably reacted with sulfuric acid to give the corresponding hydrogensulfates (Xⁿ⁻=HSO₄⁻). The transprotonation is preferably carried out using 100% strength H₂SO₄. The molar ratio of H₂SO₄ to SO₄²⁻ is preferably ≧1:1 and is, for example, in a range from 1:1 to 2:1.

In a further embodiment, the anion exchange in step b) is effected by reaction with a metal salt. This reaction is preferably carried out in a solvent from which the metal sulfate formed from the metal of the metal salt and the sulfate anion crystallizes out. The above-described hydrogensulfates can also be used for this variant of the anion exchange. The cation of the metal salt is preferably an alkali metal, alkaline earth metal, lead or silver ion. The anion of the metal salt is selected from among the abovementioned anions Xⁿ⁻, in particular anions other than Cl⁻, Br⁻, I⁻ and monoalkylsulfate and monoalkylphosphate. In a suitable procedure, a solution of the metal salt is brought into contact with a solution of the quaternary ammonium compound. Suitable solvents are, for example, water, water-miscible solvents, for example alcohols such as methanol and ethanol, and mixtures thereof. The reaction temperature is preferably in a range from −10 to 100° C., in particular from 0 to 80° C.

In a further embodiment, the anion exchange in step b) is effected by ion exchange chromatography. The basic ion exchangers which are known to those skilled in the art and comprise at least one base immobilized on a solid phase are in principle suitable for this purpose. The solid phase of these basic ion exchangers comprises, for example, a polymer matrix. Such matrices include, for example, polystyrene matrices comprising styrene together with at least one crosslinking monomer, e.g. divinylbenzene, and also, if appropriate, further comonomers in copolymerized form. Also suitable are polyacrylic matrices which are obtained by polymerization of at least one (meth)acrylate, at least one crosslinking monomer and, if appropriate, further comonomers. Suitable polymer matrices are also phenol-formaldehyde resins and polyalkylamine resins obtained, for example, by condensation of polyamines with epichlorohydrin.

The anchor groups (whose loosely bound counterions can be replaced by ions bearing a charge of the same sign) bound directly or via a spacer group to the solid phase are preferably selected from among nitrogen-containing groups, preferably tertiary and quaternary amino groups.

Suitable functional groups are, for example (in order of decreasing basicity):

| | |
|---|---|
| —CH₂N⁺(CH₃)₃ OH⁻ | e.g. Duolite A 101 |
| —CH₂N⁺(CH₃)₂CH₂CH₂OH OH⁻ | e.g. Duolite A 102 |
| —CH₂N(CH₃)₂ | e.g. Amberlite IRA 67 |
| —CH₂NHCH₃ | |
| —CH₂NH₂ | e.g. Duolite A 365 |

Both strongly basic and weakly basic ion exchangers are suitable for the process of the invention but preference is given to strongly basic ion exchangers in OH form. Among the weakly basic ion exchangers, preference is given to those bearing tertiary amino groups. Strongly basic ion exchangers generally have quaternary ammonium groups as anchor groups. Commercially available ion exchangers suitable for the process of the invention include, for example, Amberlyst® A21 (dimethylamino-functionalized, weakly basic) and Amberlyst® A27 (quaternary ammonium groups, strongly basic) and Ambersep® 900 OH (strongly basic). For the ion exchange, the ion exchangers are firstly loaded with the desired anions Xⁿ⁻ and subsequently brought into contact with the ionic compounds based on sulfate anions (or hydrogensulfate anions).

In a further embodiment, the anion exchange in step b) is effected by electrolysis (electrodialysis). The use of electrolysis cells having ion-exchange membranes thus allows, for example, the preparation of bases from the corresponding salts. Suitable electrodialysis cells and membranes for the anion exchange and also bipolar membranes for the simultaneous exchange of cations and anions are known and commercially available (e.g. from FuMA-Tech St. Ingbert, Germany; Asahi Glass; PCA-Polymerchemie Altmeier GmbH und PCCell GmbH, Lebacher Straβe 60, D-66265, Heusweiler, Germany). In this embodiment, too, the fact that quaternary ammonium compounds having polyvalent, noncorrosive anions are formed in step a) of the process of the invention is found to have a particularly advantageous effect on the life of the electrolysis apparatuses used, especially the membranes.

A first group of suitable electrolysis cells for the anion exchange are cells in which the electrode compartments are separated from one another by a membrane. Suitable membranes are, for example, membranes based on perfluoropolymers. Further suitable electrolysis cells for the anion exchange are ones in which the electrode compartments are not separated from one another by a membrane. These include, for example, “capillary gap cells” (CGC) which comprise, for example, a bipolar stack of electrode discs comprising, for example, graphite or graphite-modified polymers. Solid polymer electrolyte (SPE) cells which require no additional electrolyte are also suitable.

In an embodiment of the process for preparing quaternary ammonium hydroxides, a quaternary ammonium compound containing sulfate anions obtained by step a) of the process of the invention can, for example, be converted electrolytically into the corresponding quaternary ammonium hydroxide. If desired, the electrolytic anion exchange can be followed by ion exchange chromatography. This makes it possible to obtain highly pure quaternary ammonium compounds which obtain only extremely low concentrations or concentrations below the detection limit of undesirable anions.

The process of the invention advantageously makes it possible to prepare compounds of the general formula b Cat^{m+}×Xⁿ (I), as defined above, which are free of Cl⁻, Br⁻, I⁻ and at the same time free of monoalkylsulfate anions. To prepare compounds of the formula I having an extremely low residual content of halide ions, the reaction in steps a) and b) is preferably carried out with the exclusion of halide ions and of materials which release these. Thus, reagents, solvents, inert gases, etc., which are substantially free of halide ions can be used for the reaction. Such components are commercially available or can be prepared by customary purification methods known to those skilled in the art. These include, for example, adsorption, filtration and ion exchange processes. If desired, the apparatuses used in steps a) and b) can also be freed of halide ions before use, e.g. by rinsing with halide-free solvents. The process of the invention makes it possible to obtain compounds of the general formula I in which Xⁿ⁻ is OH⁻ and the total content of halide ions is not more than 100 ppm, preferably not more than 10 ppm and in particular not more than 1 ppm. Furthermore, it is possible to obtain compounds which have a total content of monoalkyl sulfate anions of not more than 100 ppm, preferably not more than 10 ppm and in particular not more than 1 ppm.

The invention further provides a process as defined above for preparing N,N-dimethylpiperidinium pentaborate, wherein
a) N-methylpiperidine is reacted with dimethyl sulfate at ambient pressure to give N,N-dimethylpiperidinium sulfate, and
b) the N,N-dimethylpiperidinium sulfate obtained in step a) is subjected to a single-stage or multistage anion exchange to replace the sulfate anions by pentaborate anions.

With regard to useful and preferred process conditions in steps a) and b), what has been said above about these steps is incorporated by reference at this point.

In step a), the N-methylpiperidine is preferably initially brought into contact with the dimethyl sulfate at a temperature of not more than 35° C. For example, the N-methylpiperidine in an aqueous medium, preferably water, can be placed in a reaction vessel and the dimethyl sulfate can be added with the temperature being controlled. The temperature can be kept in the desired range by addition of the dimethyl sulfate a little at a time and/or by cooling. This is preferably followed by a reaction with heating to a temperature in the range from 60 to 100° C. In an appropriate embodiment, the reaction mixture is for this purpose refluxed in an apparatus which is not closed in a pressure-tight manner and is provided with a condensation facility.

According to this process, the N,N-dimethylpiperidinium sulfate obtained in step a) is preferably firstly reacted with barium hydroxide in an aqueous medium. This results in precipitation of barium sulfate and formation of N,N-dimethylpiperidinium hydroxide which can subsequently be converted into the pentaborate by reaction with boric acid. The N,N-dimethylpiperidinium pentaborate obtained in step b) can have various hydrate contents. It can be represented by the general formula
[N,N-dimethylpiperidinium]⁺[B₅O₈]⁻×H₂O
where w is an integer or fraction from 0 to 20. The hydrate content can be controlled via the drying conditions. After sufficiently long drying, preferably at elevated temperature and under reduced pressure, the following hemihydrate (0.5H₂O) can be obtained as preferred embodiment:
[N,N-dimethylpiperidinium]⁺[B₅O₆(OH)₄]⁻×0.5H₂O
[N,N-dimethylpiperidinium]⁺[B₅O₁₀H₄]⁻×0.5H₂O

The process of the invention makes it possible to prepare N,N-dimethylpiperidinium pentaborate having a total content of halide ions of not more than 100 ppm, preferably not more than 10 ppm and in particular not more than 1 ppm.

The invention is illustrated by the following nonrestrictive examples.

### EXAMPLES

### Example 1

a) Preparation of N,N-dimethylpiperidinium sulfate by reaction in water (under ambient pressure)
   172.9 ml of distilled water and 31.7 g (0.252 mol) of N-methylpiperidine were placed in a 250 ml flask provided with a dropping funnel, reflux condenser and magnetic stirrer and not sealed of pressure-tight from the environment, and 15.1 g (0.12 mol) of dimethyl sulfate were added while stirring, with the internal temperature being kept below 30° C. by cooling in ice. The reaction mixture was subsequently refluxed for 15 hours (final temperature: 97.5° C.). The solution obtained in this way was evaporated on a rotary evaporator and the residue obtained was dried at 50° C. in an oil pump vacuum and subsequently stirred with 300 ml of acetone at room temperature for 1.5 hours. The acetone was then separated off with suction, the resulting solid was washed with 100 ml of acetone and subsequently dried in an oil pump vacuum. This gave 40.15 g of N,N-dimethylpiperidinium sulfate having a water content of 14.3%. This corresponds to a yield of 89% of theory.
b) Preparation of N,N-dimethylpiperidinium hydroxide by reaction with barium hydroxide
   32.07 g (0.1017 mol) of barium hydroxide (octahydrate) and 258.3 g of water were placed in a 500 ml round-bottomed flask provided with a dropping funnel and the mixture was heated to 40° C. 38.5 g (0.1017 mol) of the aqueous solution of the sulfate prepared in step a) was added dropwise from the dropping funnel over a period of 30 minutes. Immediately after commencement of the addition, a snow-white finely pulverulent precipitate of barium sulfate formed. After the addition was complete, the reaction mixture was stirred at 40° C. for another 7 hours, cooled and the precipitate was filtered off with suction through a blue band filter. This gave 274 g of a clear, colorless solution of N,N-dimethylpiperidinium hydroxide. Titration of the solution with 0.1 HCl indicated a hydroxide number of 8.45%, corresponding to a yield of 89% of theory. The sulfate concentration was <1 ppm.
c) Preparation of N,N-dimethylpiperidinium hydroxide over an ion exchanger laden with hydroxyl groups
   70 g of a 10% strength by weight aqueous solution of N,N-dimethylpiperidinium sulfate, obtainable by dilution of the aqueous solution prepared in step a) with deionized water, were admixed with 60 ml of a strongly basic ion exchanger (Ambersep® 900 OH) in the OH form (loading: 0.59 eq/l), and the mixture was shaken at room temperature for 24 hours. The ion exchanger was subsequently filtered off. This gave an about 10% strength by weight aqueous solution of N,N-dimethylpiperidinium hydroxide. Analysis of the solution for sulfate indicated a sulfate content of <1 ppm.
d) Preparation of N,N-dimethylpiperidinium pentaborate [N,N-dimethylpiperidinium]⁺[B₅O₁₀H₄]⁻×0.5H₂O
   2600 g of an aqueous solution of N,N-dimethylpiperidinium hydroxide, which according to titration of the OH⁻ ions contained 175.5 g (1.34 mol) of N,N-dimethylpiperidinium hydroxide, were placed in a reaction vessel at room temperature. 1710 g of water and subsequently 415 g (6.7 mol) of boric acid were then added to the reaction mixture. The mixture was stirred at room temperature for 2 hours. The conversion is quantitative.
   472.6 g of the solution obtained were dried at 40° C. on a rotary evaporator (15 mbar) for 7.5 hours and N₂ was subsequently admitted into the rotary evaporator. The product was obtained in the form of white, nonhygroscopic crystals.

Elemental analysis: (theory: 341 g/mol)

Theory: 24.63% (C); 6.16% (H); 4.11% (N); 15.84% (B)

found: 24.7% (C); 6.1% (H); 4.1% (N); 15.9% (B)

A DSC (differential scanning calorimetry) measurement indicated half an equivalent of water which was not chemically bound.

1. A process for preparing a quaternary ammonium compound, which comprises
a) reacting an amine compound which comprises at least one sp³-hybridized nitrogen atom and has a boiling point under normal conditions of at least 80° C. with a dialkyl sulfate at ambient pressure with participation of both alkyl groups of the dialkyl sulfate to give a quaternary ammonium compound containing sulfate anions, and
b) if appropriate, subjecting the quaternary ammonium compound obtained in step a) to an anion exchange.

2. The process according to claim 1, wherein the quaternary ammonium compound obtained in step a) is additionally subjected to at least one work-up step to separate off unreacted amine compound.

3. The process according to claim 1, wherein an amine compound which forms a low-boiling azeotrope with water is used in step a).

4. The process according to claim 1, wherein the amine compound used in step a) is among a compound[s] of the formula NR¹R²R³, where R¹ R² and R³ are selected independently from the group consisting of hydrogen, alkyl, cycloalkyl, heterocycloalkyl, aryl and hetaryl, with at least two of the radicals R¹ R² and R³ together with the N atom to which they are bound also being able to be part of a polycyclic compound.

5. The process according to claim 1, wherein the amine compound used in step a) is a tertiary amine.

6. The process according to claim 1, wherein the quaternary ammonium compound obtained comprises at least one anion Xⁿ⁻, where n is an integer corresponding to the valence of the anion and is selected from among OH⁻, HSO₄⁻, NO₂⁻, NO₃⁻, CN⁻, OCN⁻, NCO⁻, SCN⁻, NCS⁻, PO₄³⁻, HPO₄², (H₂PO₄⁻), H₂PO₃⁻, HPO₃², BO₃³⁻, (BO₂)₃³⁻, B₅O₆⁻, B₅O₈⁻, B₅H₄O₁₀⁻, [BF₄]⁻, [BCl₄]⁻, [B(C₆H₅)₄], [PF₆]⁻, [SbF₆]⁻, [AsF₆]⁻, [AlCl4]⁻, [AlBr₄]⁻, [ZnCl₃]⁻, dichlorocuprates (I) and (II), CO₃²⁻, HCO₃⁻, F⁻, (R′—COO)⁻, R′₃SiO⁻, (R′—SO₃)⁻ and [(R′—SO₂)₂N], where R′ is alkyl, cycloalkyl or aryl.

7. The process according to claim 1, wherein the quaternary ammonium compound obtained comprises at least one boron-containing anion.

8. The process according to claim 7, wherein the boron-containing anion is of the general formula II
[MₓB_{y}O_{z}(A)ᵥ]ⁿ⁻.wH₂O (II)
where
M is hydrogen, NH₄ or a different agriculturally acceptable cation,
A is a ligand,
n is an integer in the range from 1 to 6,
x is an integer or fraction in the range from 0 to 10,
y is an integer or fraction in the range from 1 to 48,
z is an integer or fraction in the range from 0 to 48,
v is an integer or fraction in the range from 0 to 24, and
w is an integer or fraction in the range from 0 to 24.

9. The process according to claim 1, wherein the reaction in step a) is carried out at a temperature in the range from 40 to 120° C.

10. The process according to claim 1, wherein, in step a), the amine compound is initially brought into contact with the dialkyl sulfate at a temperature of not more than 35° C. and the resulting mixture is subsequently heated to a temperature of at least 40° C.

11. The process according to claim 1, wherein the amine compound and the dialkyl sulfate are used in a molar ratio of at least 2:1 in step a).

12. The process according to claim 1, wherein the reaction in step a) is carried out in an aqueous solvent.

13. The process according to claim 1, wherein the reaction in step a) is carried out in the presence of an inert gas.

14. The process according to any of the preceding claim 1, wherein the process steps a) and b) are carried out in the absence of halide ions.

15. The process according to claim 1, wherein the anion exchange in step b) is effected by transprotonation, reaction with a metal salt, ion exchange chromatography, electrolytically or a combination thereof.

16. The process according to claim 15, wherein the reaction with the metal salt is carried out in a solvent from which a metal sulfate formed from the metal of the metal salt and the sulfate anion crystallizes out.

17. A process for preparing N,N-dimethylpiperidinium pentaborate, wherein
a) N-methylpiperidine is reacted with dimethyl sulfate at ambient pressure to give N,N-dimethylpiperidinium sulfate, and
b) the N,N-dimethylpiperidinium sulfate obtained in step a) is subjected to a single-stage or multistage anion exchange to replace the sulfate anions by pentaborate anions.

18. The process according to claim 17, wherein, in step b), the N,N-dimethylpiperidinium sulfate is firstly reacted with barium hydroxide in an aqueous medium to produce N,N-dimethylpiperidinium hydroxide and the N,N-dimethylpiperidinium hydroxide so obtained is subsequently reacted with boric acid.

19. The process according to claim 5, wherein the amine is a tertiary cyclic amine.

20. The process according to claim 19, wherein the amine is N-methylpiperidine.

21. The process according to claim 9, where the temperature is from 60 to 100° C.
